(19) United States
(12) Patent Application Publication
(10) Pub. No.: US 2016/0000986 A1
(43) Pub. Date: Jan. 7, 2016

(54) BLOOD OXYGENATION SYSTEM

(71) Applicant: Fouad Samir Said, Los Angeles, CA (US)

(72) Inventor: Fouad Samir Said, Los Angeles, CA (US)

(21) Appl. No.: 14/321,057

(22) Filed: Jul. 1, 2014

(51) Int. Cl. A61M 1/32

(52) U.S. Cl. CPC A61M 1/32

(57) ABSTRACT

A blood oxygenation system which prevents transmigration of blood to gaseous oxygen circuitry. The system includes a blood circuit, an oxygen supply circuit, an oxygen-blood interface in which gaseous oxygen is absorbed into the blood, and an oxygen disposal circuit for disposing of unused oxygen. The oxygen-blood interface is arranged to prevent transmigration of blood into the oxygen circuit. The blood circuit includes a pump and a blood de-aerator. The oxygen supply circuit includes an oxygen supply, an ozone generator, and an ozone destructor. A silica gel trap in the oxygen disposal circuit separates O₂ from ozone prior to the latter entering the ozone destructor.

### FIELD OF THE INVENTION

The present invention relates to medical blood treatment, and more particularly, to a blood treatment system for oxygenating the blood.

### BACKGROUND OF THE INVENTION

Benefits of oxygenating the blood have come to the notice of the medical community. Accordingly, systems have been proposed for oxygenating the blood by removing blood from a patient's body, oxygenating the blood, and returning oxygenated blood to the body. It is not necessarily easy to dissolve all oxygen sources, especially since oxygen being added to the blood is in gaseous form, in the blood. There exists the possibility of blood escaping a blood circuit and entering an oxygen circuit. Blood oxygenation systems are in need of a system which prevents such an occurrence.

### SUMMARY OF THE INVENTION

The present invention addresses the above stated situation by providing a blood oxygenation system which prevents transmigration of blood to gaseous circuitry. The system includes a blood circuit in which blood is extracted from the patient's body pumped, oxygenated, de-aerated, and returned to the body, and an oxygen circuit. The oxygen circuit includes an oxygen supply, an ozone generator fed oxygen from the oxygen supply, and an oxygen gas waste portion for discharging oxygen not absorbed in the blood. The oxygen circuit supplies both O₂ and O₃ in gaseous form. A blood-oxygen interface receives unoxygenated blood from the blood circuit and discharges oxygenated blood to the blood circuit. The blood-oxygen interface is provided with oxygen from the oxygen circuit. The blood-oxygen interface is managed so as to prevent blood from entering the oxygen gas supply and the oxygen gas waste portion.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a schematic diagram showing major components of a blood oxygenation system, according to at least one aspect of the invention;

FIG. 2 is an enlarged detail view of a component seen at the lower center of FIG. 1;

FIG. 3 is an enlarged detail view of an object which may be incorporated in the component shown in FIG. 2; and

FIG. 4 is an enlarged detail view of an alternative to the component of FIG. 2.

### DETAILED DESCRIPTION

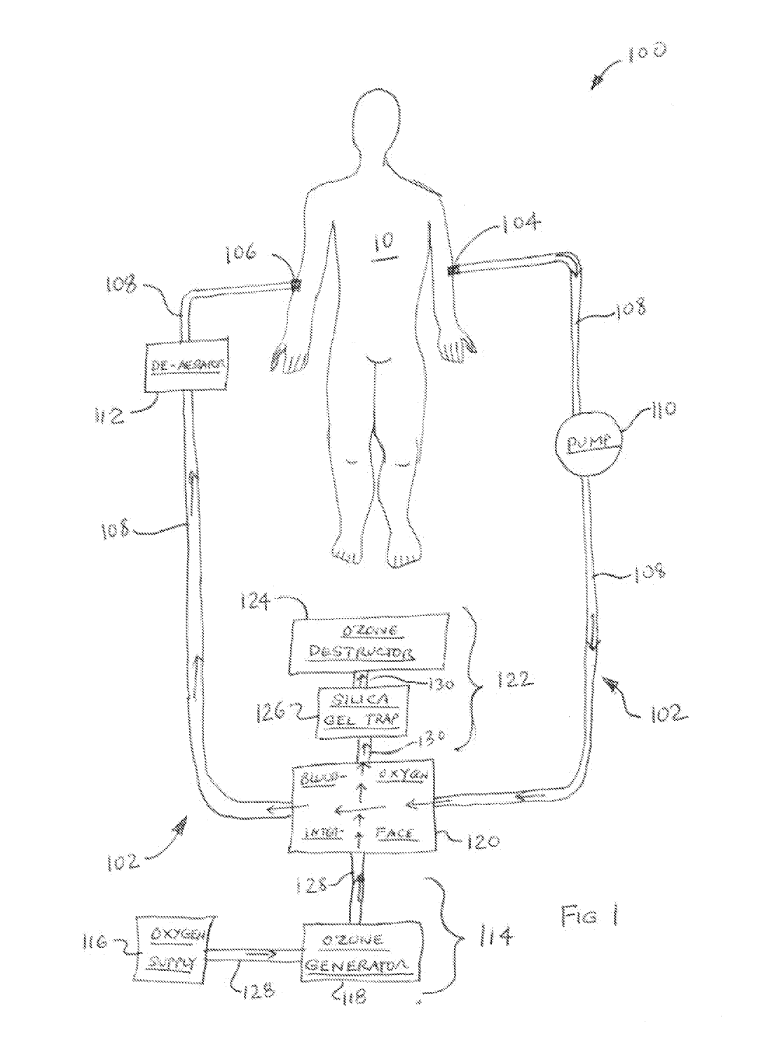

Referring first to FIG. 1, according to at least one aspect of the invention, there is shown a blood oxygenation system 100 for infusing blood of a patient 10 with oxygen and returning oxygenated blood to the patient 10. The blood oxygenation system 100 comprises a blood circuit 102 including an extraction port 104 for obtaining blood from the patient 10, a return port 106 for returning oxygenated blood to the patient 10, a blood conduit 108 connected to the extraction port 104 and the return port 106, a blood pump 110 serially located in the blood conduit 102, and a de-aerator 112 for separating air from oxygenated blood, located in the blood circuit 102 prior to the blood return port 106.

The blood oxygenation system 100 comprises an oxygen supply circuit 114 including an oxygen supply 116, and an ozone generator 118 fed oxygen from the oxygen supply 116.

The blood oxygenation system 100 comprises a blood-oxygen interface 120 which dissolves into the blood gaseous oxygen supplied from the oxygen supply circuit 114.

The blood oxygenation system 100 comprises an oxygen disposal circuit 122 for discharging oxygen not absorbed by the blood in the blood-oxygen interface 120.

The blood-oxygen interface 120 prevents transmigration of blood into the oxygen supply circuit 114 and into the oxygen disposal circuit 122.

The extraction port 104 and the return port 106 may each be any known long term or short term venous catheter having a sharp pointed needle, for example, to facilitate penetration of the skin and an artery or vein.

The oxygen disposal circuit 122 further comprises an ozone destructor 124 and an oxygen-ozone separator 126, with only ozone being conducted to the ozone destructor 124. The oxygen-ozone separator 124 comprises a silica gel trap.

The oxygen supply circuit 114 comprises a conduit 128 which conducts oxygen to the ozone generator 118 from the oxygen supply 116, and which conducts oxygen and ozone from the ozone generator 118 to the blood-oxygen interface 120.

The oxygen disposal circuit 122 comprises a conduit 130 which conducts unused oxygen, in both O₂ and ozone form, from the blood-oxygen interface 120 first to the oxygen-ozone separator 126, then to the ozone destructor 124 for final disposal.

In FIGS. 1, 2, and 4, direction of flow is indicated by arrows within the conduits 108, 128, and 130. Hence description of the de-aerator 112 as being located in the blood circuit 102 prior to the blood return port 106 will be understood within the context of direction of flow within the conduit 108.

The the pump 110, the de-aerator 112, the oxygen supply 116, the ozone generator 118, the oxygen-ozone separator 126, and the ozone destructor 124 are shown in simplified or schematic form only, as these components are themselves may be conventional. For example, these components may have the structure of their corresponding components shown in any of the systems described in U.S. Pat. No. 5,052,382, to Wainwright; U.S. Pat. No. 5,540,898, to Davidson; U.S. Pat. No. 6,007,785, to Liou; U.S. Pat. No. 6,027,688, to Wainwright; U.S. Pat. No. 6,073,627, to Sunnen; and U.S. Pat. No. 6,736,788, to Mongomery, respective contents of which are hereby incorporated herein by reference as though fully reproduced herein. components such as the ozone generator 118 for example will be understood to include all necessary inputs and connections, such as electrical power, for operability.

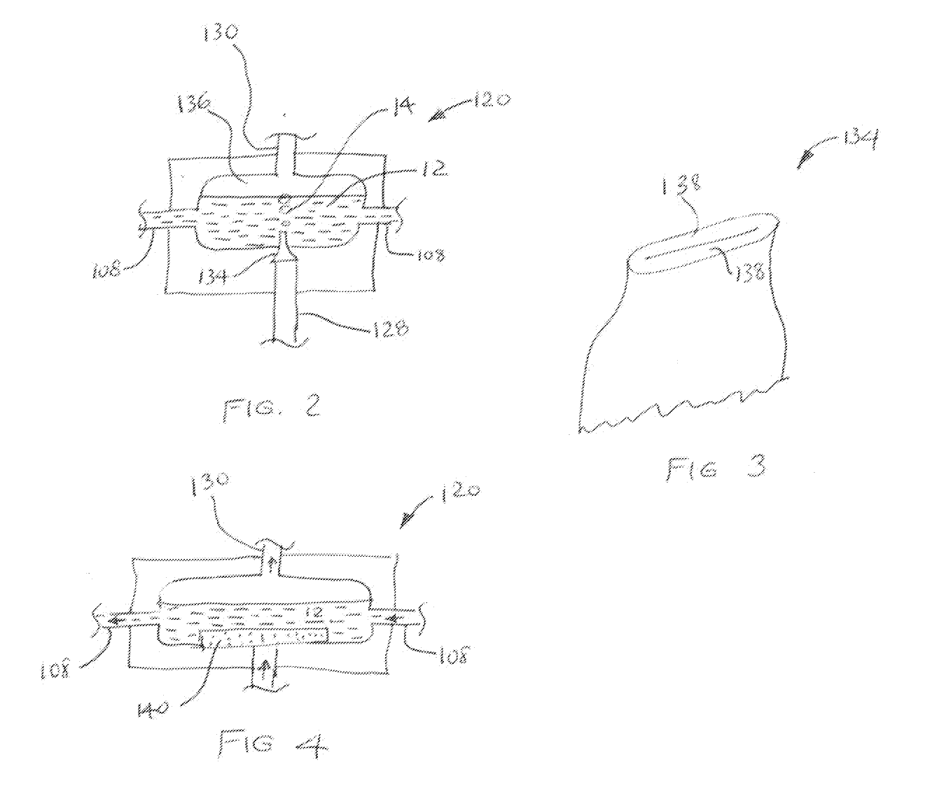

Referring now to FIGS. 2 and 3, the blood oxygen interface 120, in order to prevent transmigration of blood into the oxygen supply circuit 114 and into the oxygen disposal circuit 122, may comprise a one-way valve 134 connected to the conduit 128 and operable to discharge oxygen bubbles 14 into and through the blood 12. Oxygen not absorbed by the blood 12 may collect in a headspace 136 by gravity and then pass to the conduit 130. The one-way valve 134 may be for example, a rubbery or flexible hose which, in the absence of a predetermined level of pressure in the conduit 128, is biased to remain closed, as seen in FIG. 3. Above the predetermined level, oxygen forces the one-way valve 134 to expand slightly, thereby causing a gap (not shown) to form at normally closed, abutting lips 138.

In an alternative to the pressure responsive one-way valve 134, and turning now to FIG. 4, the blood-oxygen interface 120 may include a selectively permeable membrane 140, which passes oxygen gases (i.e., O2 and ozone), but does not permit permeation by the blood 12. A selectively permeable membrane (not shown) may be provided to facilitate separation of oxygen gases where the headspace 136 is shown, for example.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A blood oxygenation system for infusing blood of a patient with oxygen and returning oxygenated blood to the patient, comprising:
a blood circuit including an extraction port for obtaining blood from the patient, a return port for returning oxygenated blood to the patient, a blood conduit connected to the extraction port and the return port, a blood pump serially located in the blood conduit, and a de-aerator for separating air from oxygenated blood, located in the blood circuit prior to the blood return port;
an oxygen supply circuit including an oxygen supply, and an ozone generator fed oxygen from the oxygen supply;
a blood-oxygen interface which dissolves into the blood gaseous oxygen supplied from the oxygen supply circuit; and
an oxygen disposal circuit for discharging oxygen not absorbed by the blood in the blood-oxygen interface, wherein
the blood-oxygen interface prevents transmigration of blood into the oxygen supply circuit and into the oxygen disposal circuit.

2. The blood oxygenation system of claim 1, wherein the oxygen disposal circuit further comprises an ozone destructor.

3. The blood oxygenation system of claim 2, wherein the oxygen disposal circuit further comprises an oxygen-ozone separator, with only ozone being conducted to the ozone destructor.

4. The blood oxygenation system of claim 3, wherein the oxygen-ozone separator comprises a silica gel trap.
